(19) United States
(12) Patent Application Publication
(10) Pub. No.: US 2017/0238875 A1
(43) Pub. Date: Aug. 24, 2017

(54) Biologically Inspired Motion Compensation and Real-Time Physiological Load Estimation Using a Dynamic Heart Rate Prediction Model

(71) Applicant: Lifeq Global Limited, Dublin (IE)

(72) Inventors: Laurence Richard Olivier, Alpharetta, GA (US); Franco Bauer du Preez, Cobham (GB)

(73) Assignee: LifeQ Global Limited, Dublin (IE)

(21) Appl. No.: 15/521,667

(22) PCT Filed: Aug. 6, 2015
(86) PCT No.: PCT/US15/43919

§ 371 Date: Apr. 25, 2017

Related U.S. Application Data
(60) Provisional application No. 62/068,882, filed on Oct. 27, 2014.

(51) Int. Cl. A61B 5/00; A61B 5/0205; G06F 19/00; A61B 5/0404

(52) U.S. Cl. CPC A61B 5/721; A61B 5/0004; A61B 5/0022; A61B 5/0404; A61B 5/0205; A61B 5/681; A61B 5/7275; A61B 5/4866; A61B 5/7278; G06F 19/3437; A61B 5/1118

(57) ABSTRACT

The current invention pertains to a method whereby the accuracy of a heart rate prediction gathered from sensor data can be improved during periods when motion corrupts the signal. The model utilized can also be inverted to infer information on the physiological state of a subject, such as real-time energy utilization or physiological load. In addition, this method can also be used to segment the contribution of each energy system, namely the phosphagen system, anaerobic glycolysis and aerobic respiration, to the physiological load experienced by the user. At the core of this approach lies a model describing the dynamic adjustment of human heart rate under varying physiological demands.

### FIELD OF THE INVENTION

The present inventions pertain to the field of non-invasive monitoring of physiological parameters. More specifically, a system and method is introduced by which the accuracy of a heart rate prediction from sensor data can be improved under conditions where movement distorts the signal. In addition, the model utilized in said method can be inverted to infer information about the physiological state of a subject, such as real-time energy utilization. At the core of this approach lies a model describing the dynamic adjustment of human heart rate under varying physiological demands.

### BACKGROUND OF THE INVENTION

The health benefit derived from tracking your heart rate over time is gaining the attention of a growing number of individuals. While there is a clear movement from chest strap based heart rate monitors to wearable solutions, it remains that the heart rate signals measured using both electrocardiography (ECG) and photoplethysmography (PPG) can become corrupted by motion artifacts during periods of physical activity. Inertial motion sensors, such as accelerometers, have become a prevalent tool for measuring motion and/or activity and therefore form part of a number of patents and applications aiming to compensate for the motion artifacts known to corrupt physiological signals. An example is described by patent application US 20120150052 to Schoshe Industries Inc. which uses a motion sensing system (a red LED) and an accelerometer to sense changes in position of an optical heart rate monitor in relation to the skin and in relation to the heart, respectively. Information from the motion sensing system and accelerometer are then used to compensate for the motion artifacts in the PPG heart rate monitor signals. Similarly, the patent application US20140018635 uses a Kalman filter to adaptively filter out the noise in the optical signal using the accelerometer signal. Other patents that describe similar methodologies include U.S. Pat. No. 8,945,017 (Fitbit Inc.) and EP 2229880 (CSEM). In addition, U.S. Pat. No. 8,483,788 describes a motion compensated pulse oximeter which uses an accelerometer to measure the changes induced by motion between the light emitter and detector. An attenuation factor is then calculated using a combination of the accelerometer data, an equation related to a model distance between light emitter and detector and a model based on the expected behavior of light. A look-up table is then used to find a motion measurement that corresponds to the attenuation factor and this measure is used to better calculate the physiological parameter of interest.

Patent application US 20140213858 to Koninklijke Philips N.V. approaches the problem by measuring the signal quality of the optical heart rate signal first. Only if the signal quality falls below a certain threshold the motion signal is used to estimate the heart rate using a exponential predictive model. Various patent applications and publications have also made use of dynamic models and modeling techniques in order to extract physiological information such as heart rate, especially in the presence of motion artifacts. For example, US patent application 20100274102 to Streamline Automation, Llc., disclose a system and method whereby physiological data from a biomedical sensor (e.g. pulse oximeter, electrocardiograph) is processed using a probabilistic model for the removal of noise and motion artifacts. The invention incorporates a dynamic state-space model (DSSM) and a data processor capable of combining a sigma point Kalman filter (SPKF) or a sequential Monte Carlo (SMC) algorithm with Bayesian statistics. In addition, a mathematical model which is constituted by a cardiovascular and photoplethysmography (PPG) model is used in order to remove noise and motion artifacts.

The current invention, which is explained in more detail below, proposes a dynamic heart rate model which can predict heart rate changes based on an inferred activity level. This is to be used in situations where the heart rate cannot be separated from the motion signal during exercise and therefore provides a smooth crossing. The model is probabilistic and maps the heart rate trajectory to physiological load. In this way, an inverse version of the model can also be used to predict physiological load. This shows energy expenditure in a more responsive manner than what has been considered in the current state of the art. For example (WO 201412083, WO 201008443, EP 2489302, WO 2012172375), present methodologies for the estimation of energy expenditure during exercise, however these are unlike the current invention and lack the ability to distinguish between the three energy systems from which energy supply is derived.

The energy requirements of muscle are fulfilled by three energy systems: the anaerobic energy system, further classified into alactic and lactic components and the aerobic energy system. Exercise segmentation thus refers to the determination of the relative contribution of each of the three energy systems to the total energy supply during exercise. The current state of the art regarding exercise segmentation is somewhat reliant on the determination of anaerobic and/or aerobic thresholds, which tends to yield inaccurate assumptions regarding the relative contribution of each of these energy systems as well as the times course and extent to which they are utilized during exercise.

For example U.S. Pat. No. 5,810,722, to Polar Electro Oy., disclose a system and method whereby the aerobic and anaerobic thresholds can be determined. The fundamental premise of the approach includes subjecting an individual to a progressively increasing stress (i.e exercise intensity) to obtain threshold values for aerobic and anaerobic metabolism. The approach taken is based on ECG readings and the threshold values are determined on the basis of heart rate and respiratory frequency data obtained from the ECG sensor. In line with this patent, the methodology presented by patent application CA 2656538 involves the determination of metabolic transition points by calculating respiratory rate (RR), heart rate (HR) and the ratio of RR:HR at more than one time point during a task, thus describing the metabolic transition points as identifiable points of time of the RR:HR ratio. Some inventions have used measures of respiratory exchange ratio (RER) and heart rate to determine the anaerobic threshold (U.S. Pat. No. 7,390,304, U.S. Pat. No. 5,297,558, U.S. Pat. No. 6,554,776) whilst others have estimated the aerobic and anaerobic threshold based on heart rate zones (WO 1996020640).

Another approach, taken by both EP 1127543 and EP 1125744 to Polar Electro Oy., makes use of a mathematical model to determine the lactate concentration. The mathematical model is implemented as a neural network whereby heart rate data is related to lactate concentration as determined by a stress level, with reference being made to aerobic and anaerobic reactions (energy metabolism) as well as glucose. Furthermore, the invention of US patent publication 50187626 makes use of a mathematical model whereby anaerobic capacity is determined by analysis of the logarithmic decay of the derived power values (i.e the time taken to fully deplete a logarithmic function that approximates the derived power value is taken as the anaerobic capacity value). Thus, this approach is largely based on power output and maximal exertion.

Patent publication U.S. Pat. No. 6,920,348 describes the analysis of ECG measurements (namely Wilson points) in order to determine metabolic factors. Metabolic factors are determined using a first derivative of an ECG measurement, determining an absolute value of a positive spike of a first derivative (Rx), a sum of absolute values of the positive and negative spikes of the first derivative (RSx) and by dividing Rx by RSx in order to determine a number proportional to the metabolic factor (Vx). Metabolic factors included in the invention are aerobic capacity, lactic demia (anaerobic power and capacity), phosphocreatine capacity (anaerobic capacity), total metabolic capacity and total anaerobic capacity.

Patent application EP 2815344 discloses a system and method in which a data based modeling technique (relating heart rate response to exercise intensity) is configured to estimate and predict lactate threshold which can be used to predict and/or monitor the transition between aerobic and anaerobic training zones. Lastly, in patent application EP2705791 to Toumaz Healthcare, a system is described for estimating aerobic and anaerobic energy levels in order to detect the point at which a subject reaches the so-called lactate threshold, thereby allowing for adjustment in energy consumption predictions using this knowledge. At the lactate threshold, the energy production comprises both aerobic and anaerobic energy production, which have vastly different efficiencies, whilst below this threshold, only aerobic energy production is considered, which simplifies these calculations. The existence of this patent highlights the need for segmenting estimations of human energy consumption in terms of the energy systems that are involved. In the case of patent EP2705791 this segmentation is done with regards to the lactate threshold, which is defined with respect to steady state energy consumption in the body. For example, an athlete running below his lactate threshold can maintain a purely aerobic energy consumption.

While the above mentioned approaches all provide novel inventions regarding the estimation of the transitions between the aerobic and anaerobic energy systems, with most being dependent on the lactate threshold, none of these provide a solution which incorporates what is known about the physiology of the three energy systems. For example, although each system can be viewed as separate entity they are closely integrated and function together in order to ensure the sufficient supply and regeneration of adenosine triphosphate (ATP), a high energy phosphate molecule responsible for providing energy for all biological work. It is important to note that the three energy systems are not activated sequentially as they do not operate in discrete time periods. Rather, all physical activities will derive some energy from each of the three systems, however their relative contributions are dependent on the duration and intensity of a specific exercise bout or session.

### SUMMARY OF THE INVENTION

The current invention is comprised of three areas, namely heart rate (HR) prediction accuracy, real-time energy utilization and energy system segmentation in tandem, although it should be noted that all three approaches rely on similar or the same underlying model that describes dynamic changes in HR under different physiological demands. Physiological load is defined here as the total amount of energy demanded and supplied by the body of a subject. This quantity can be expressed in standard units of energy, such as the Watt, or normalized to the maximum energy generating capacity of an individual and expressed as a percentage value. With respect to determining real-time energy expenditure and its segmentation in terms of different biochemical energy systems (phosphagens/anaerobic/aerobic) this method is performed in lieu of the steady state concept, and aims to calculate and segment energy consumption in terms of instantaneous activity levels for these systems. One of the outcomes of this approach is that even a sub-lactate threshold exercise session will show an initial phase of anaerobic energy utilization before aerobic energy systems are activated to a sufficient level to fully match the subject's steady-state energy demands.

HR Prediction Accuracy Using a Dynamic Heart Rate Model:

As highlighted in the background section, many sensor technologies used to estimate HR, suffer losses in accuracy due to motion artifacts. Motion artifacts can be further divided into periodic and non-periodic, where many common exercise modalities generate periodic noise. With the wide availability of Microelectromechanical systems (MEMs) devices capable of providing acceleration and gyroscope readings, it is possible to obtain an independent measurement of motion artifacts that can be used to aid interpretation of the channel from which the heart rate is estimated, typically in the form of photoplethysmography (PPG). Periodic motion artifacts are often observed due to the cadence or foot strike rate of an athlete during an activity, and have a relatively stable frequency and intensity value per exercise modality, such as jogging. When HR increases from a resting value (termed rHR, typically 70 bpm) during an exercise session such that it catches up to and eclipses the cadence noise signal (typically 150 strides per minute for jogging), it becomes difficult to separate HR and motion artifacts when employing frequency domain based techniques such as the fast Fourier transform (FFT).

The presented system and method comprises a model that predicts HR changes based on an inferred activity level (typically from an accelerometer channel) to predict a likely HR trajectory under conditions where the HR signal can not be accurately separated from the motion artifact signal, allowing for a smooth crossing of the predicted HR and motion frequencies during exercise. Central to the technique is the assumption that a mapping exists between the accelerometer-based activity and the physiological load that the exercise places on a test subject. It is important to note that this mapping, or multiplier value, does not remain constant between different exercises and different sensor positions, but generally does so within the same exercise session where the sensor remains in the same position. Using a probabilistic model, where changes to this mapping coefficient are highly likely at exercise transitions (as determined by the accelerometer), it is possible to derive a most likely sequence of mapping coefficients and thereby physiological load, and likely HR trajectory predictions.

Real-Time Energy Expenditure:

In the process described above, a continuous estimate of physiological load is also obtained, which can be used to show energy expenditure in a more accurate and responsive fashion than what is possible when an instantaneous HR value is considered as a measure of instantaneous metabolic activity level, which is the current state of the art. In order to do this, the dynamic HR model is inverted to produce the physiological load estimate based on a given time series of HR predictions. This makes it possible to apply the model to HR predictions originating from any device producing such an output, including ECG and PPG based technologies, and to provide a measure of instantaneous physiological load. In order to describe this process, a simplified HR prediction model will be used as an example to illustrate the inversion process (see detailed description).

Real-Time Energy Segmentation:

The current invention introduces a similar secondary model, which predicts the segmentation of the physiological load into contributions from the different energy production systems. Typically the production systems include, but are not limited to, alactic anaerobic (phosphagen system), lactic anaerobic and aerobic processes. The model keeps track of the state of each of these systems, typically, but not limited to, an ordinary differential equation (ODE) model. The states of the energy production systems change in accordance with the physiological load and the substrates from which they derive energy. The alactic anaerobic process relies on high energy phosphate bonds stored in ATP, creatine-phosphate and other similar molecules. This energy system is the most direct link to muscle proteins that consume energy to produce movement and is therefore the fastest to respond to changes in energy demand. Lactic fermentation can be seen as the second link in this chain where the first regeneration of ATP occurs as part of the breakdown of sugars such as glucose. The last and least responsive link to physiological energy demand is the aerobic energy system which requires the complete oxidation of glucose molecules via the cell's mitochondria to produce a large number of ATP molecules compared to the lactic anaerobic process. This system is, however limited by the availability of oxygen and the clearance rate of carbon-dioxide molecules. The utility of predicting the contribution of each of these energy systems towards the instantaneous physiological load includes being able to provide feedback on the type of energy systems trained during bouts of different exercise durations and types in order to aid individuals in tailoring their training towards improving the energy systems of interest.

### BRIEF DESCRIPTION OF THE DRAWINGS

The preferred embodiments of the invention will be described by way of example only, with reference to the accompanying drawings:

FIG. 1: A depiction of the output from a simple model mapping physiological load to heart rate changes.

FIG. 2: A representation of the mapping of heart rate changes to physiological load and the inferred load difference that should be made during a tandem cycling and jogging session.

FIG. 3: A depiction of the different activity to physiological load mappings for data gathered from a tandem cycling and jogging session.

FIG. 4: A depiction of the corrected physiological load mappings based on the dynamic heart rate model combined with a probabilistic inference method (HMM).

FIG. 5: A representation of the intersection of periodic cadence noise with the heart rate signal.

FIG. 6: A graph showing heart rate data for two tandem jogging sessions at different exercise intensities.

FIG. 7: A representation of the inferred physiological load for the two jogging sessions of differing intensity as shown in FIG. 6.

FIG. 8: The output for a simple model of the three different energy systems under full physiological load.

FIG. 9: A representation of the application of the energy system model to the physiological load estimated in FIG. 7.

FIG. 10: A representation of the segmentation of energy utilization for the physiological load estimated in FIG. 7.

FIG. 11 shows a basic embodiment of the invention in the context of mobile and Internet technologies.

### DETAILED DESCRIPTION OF THE PREFERRED EMBODIMENTS

The following detailed description and drawings describe different aspects of the current invention. The description and drawings serve to enable one skilled in the art to fully understand the current invention and are not intended to limit the scope of the invention in any manner. Before the present methods and systems are disclosed and described, it is to be understood that the methods and systems are not limited to special methods, special components, or to particular implementations. It is also to be understood that the terminology used herein is for the purpose of describing particular aspects only and is not intended to be limiting.

The premise of the current invention is demonstrated using a simple example model. The model is defined mathematically, some of its basic behaviors are demonstrated and in addition, the novel ways in which it can be applied are also presented. The model takes some measure of physical activity level as input—in this case this is demonstrated using the readings from an accelerometer placed on the upper arm of a test subject. For this exemplary embodiment it is supposed that the maximum acceleration vector that can be measured has a magnitude that is six times the magnitude of gravitational acceleration (6G). 1G is then subtracted due to gravity, the absolute value is taken (as upward acceleration could result in negative acceleration values) and this is resealed to a percentage value of the maximum acceleration recorded over a small time window. When viewed it would be typical to see percentage values close to zero when a subject is resting, whereas a jogging subject would generate values that are typically in the tens of percents. This percentage value is termed the measured activity level (MA), and this example is stated simply for demonstration purposes to cover the general process of converting physical movement related signals into an estimate of physical activity level.

If it is assumed that there is some mapping between this measured activity level and the physiological energy demand that a subject's body experiences, the measured activity level can be converted into an inferred physiological load value. When such a load is applied to an individual's physiology, the body reacts by increasing the heart rate and heart stroke volume to the point where the amount of oxygen delivered to the muscles matches the physiological load. For a certain sustainable physiological load, an individual will have a heart rate at which the supply of oxygen and demand for metabolic energy are equally matched. In this embodiment, the target heart rate is designated as the heart rate for a specific exercise at a constant load.

Conceivable values for the target heart rate range between a minimum measured at rest (rHR) and a maximum determined at peak exercise intensity. The physiological load of an exercise can be mapped to a target heart rate (tHR), in the simplest case by simply employing a linear equation, with constant k1 such as:

tHR=k1(MA)+rHR (1)

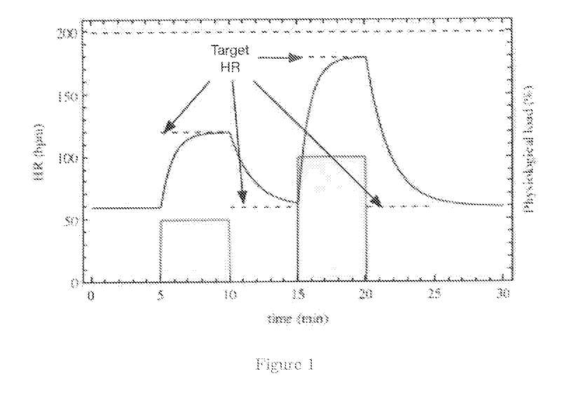

In FIG. 1 equation 1 has been employed for two exercise sessions, one at half the maximal physiological load (50%) and the next at a full physiological load (100%). The target heart rate is indicated with dashed lines for rest at 60 bpm, at 120 bpm for the first exercise session and at 180 bpm for the second exercise session.

Following this, equation 2 describes how heart rate changes in time (sHR′(t)) to reach the target heart rate. In real exercise data, the relationship resembles an exponential decay of the difference between the current heart rate and target heart rate. This can be described using an ordinary differential equation where the heart rate changes in proportion to said difference.

sHR′(t)=k2(tHR−sHR)+rHR (2)

The relaxation constant k2 of equation 2, is better described with two separate values, k2a and k2b, for instances where sHR<tHR and sHR>=tHR respectively, as the heart rate generally adapts faster to increased target HR values than decreased HR values. This provides a complete description for a simple instance of a dynamic heart rate model.

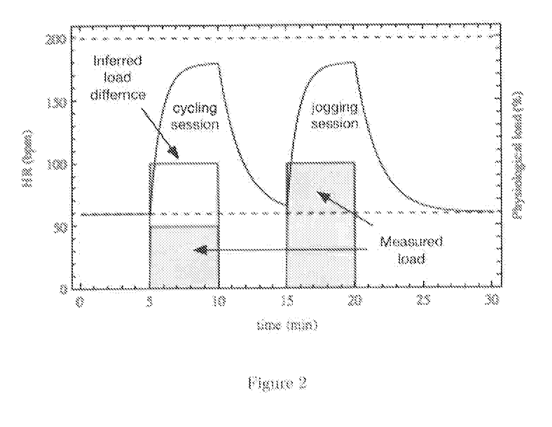

In FIG. 2 the model output for two simulated exercise sessions where the same physiological load was applied, first in a jogging and then a cycling session, is shown. In both cases the subject is faced with a full physiological load (100%) for 5 minutes, but the physical activity readings require different multipliers to arrive at 100%. In this case additional information is clearly needed in order to find the appropriate coefficient to map between the activity reading of the accelerometer and the physiological load that the subject experiences. If a gold-standard device such as an ECG heart rate monitor was used, this makes it possible to calculate the physiological load and the appropriate factor for mapping the activity measurement to heart rate, which would show a factor two difference for the time segment where the subject cycled compared to where the subject was jogging.

For applications where the sensor used to determine heart rate is susceptible to motion artifacts, such as PPG based technologies, heart rate predictions made during times of heavy signal distortion can be augmented by outputs from an accelerometer based HR prediction. Numerous statistical frameworks exist whereby noisy readings can be dramatically improved by making use of a physical model of the system and independent noise measurements. In such approaches, estimates of the internal state of the model are continually updated based on sensor readings when a clear signal is received, and the model becomes more autonomous and is relied on more heavily when the signal quality becomes poor.

One application of such a probabilistic framework could be a Hidden Markov Model, which is a statistical model containing observable quantities, as well as the hidden states of an underlying model. When combining the model discussed thus far with accelerometer readings, the activity measure and heart rate are both observables. As pointed out in FIG. 2, the mapping from physical activity measurements to the physiological load on a subject can vary significantly between different exercise modalities, but is generally similar within a session consisting of one exercise modality. The discrepancy in this mapping can be described simply as a hidden state in an HMM and the algorithms for inferring the most likely value for this discrepancy, such as the forward algorithm (for local real-time estimation) or the backward algorithm (for the most likely global estimation) are well established. Following on from this, an exemplary embodiment of how such an approach can be implemented to infer an instantaneous physiological load value for real data gathered from the cycling and running exercise session discussed earlier is provided.

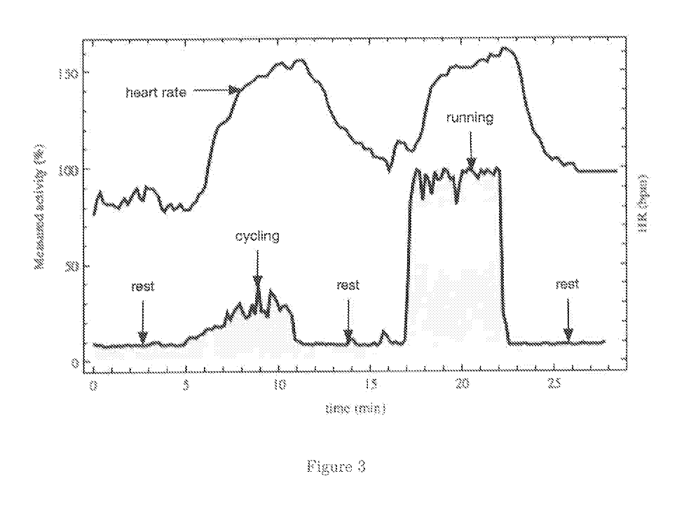

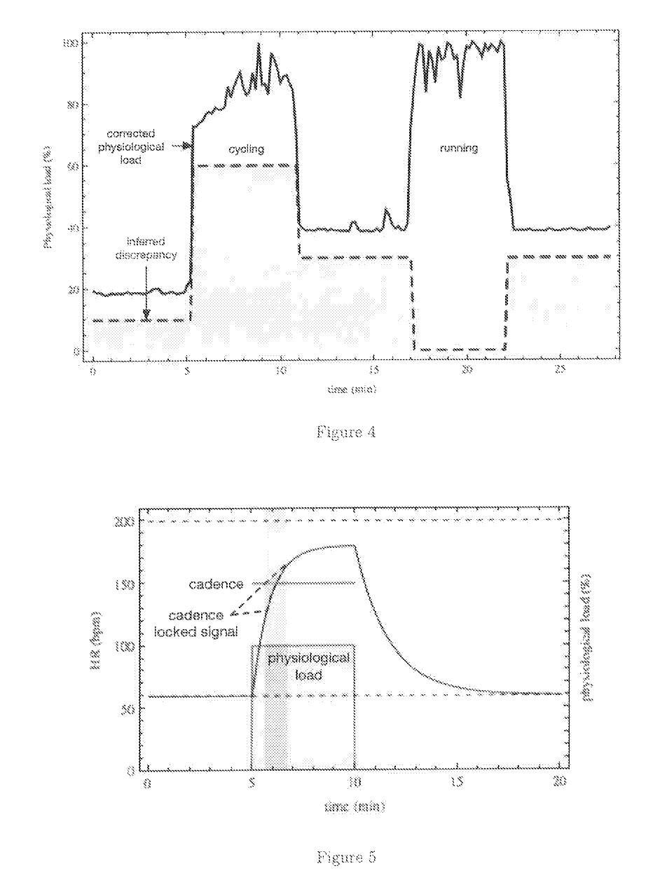

In FIG. 3 the real data gathered from an exercise and jogging session similar to the one described earlier in FIG. 2 is shown. The lower curve in FIG. 3 shows the measured activity level according to a 6G triaxial accelerometer for which the total acceleration was determined and converted as described earlier to a percentage value to indicate a measured activity level. The upper curve in FIG. 3 shows the heart rate recorded during the exercise session. From the figure it is clear that although the two exercise sessions reached similar maximum heart rate values (around 160 bpm) after 5 minutes, the measured activity values are vastly different between the two (around 30% for cycling and over 90% for running). This is expected, knowing that the test subject's arms were swinging during the run, while they were rather stationary while gripping the bicycle's handle bars. In FIG. 4 it is shown that by using the dynamic heart rate prediction model discussed earlier together with the activity measurement added to the activity discrepancy state modeled in the HMM outlined above, it is possible to obtain a realistic physiological load value for both exercises sessions (around 85% for cycling and around 95% for jogging). The discrepancy curve also highlights the slightly elevated physiological load between and after exercise sessions, which can be attributed in part to a phenomenon known as Excess Post-Exercise Oxygen uptake (EPOC), whereby anaerobic energy systems are recharged to normal levels after exercise (Ie. the phosphagen system and lactic fermentation system). A more in depth analysis of these systems is provided in the next section.

In addition to the hidden states chosen above to infer the true physiological load of an exercise, it is also possible to model hidden states wherein the motion distortion signal and heart rate signal are expected to occur at such similar frequencies that they cannot be separated from each other during signal processing (FIG. 5) when using commonly employed frequency domain methods such as the Fast Fourier Transform (FFT). These temporary situations are termed ‘cadence locks’ and by following only the accelerometer-based HR prediction during this period, a best guess of the likely heart rate trajectory can be provided. This predicted HR can also be used to improve detection of the first clearly measured HR reading after exiting this cadence lock state. Note that the accelerometer is used for obtaining both a measure of activity level and the running cadence in this example.

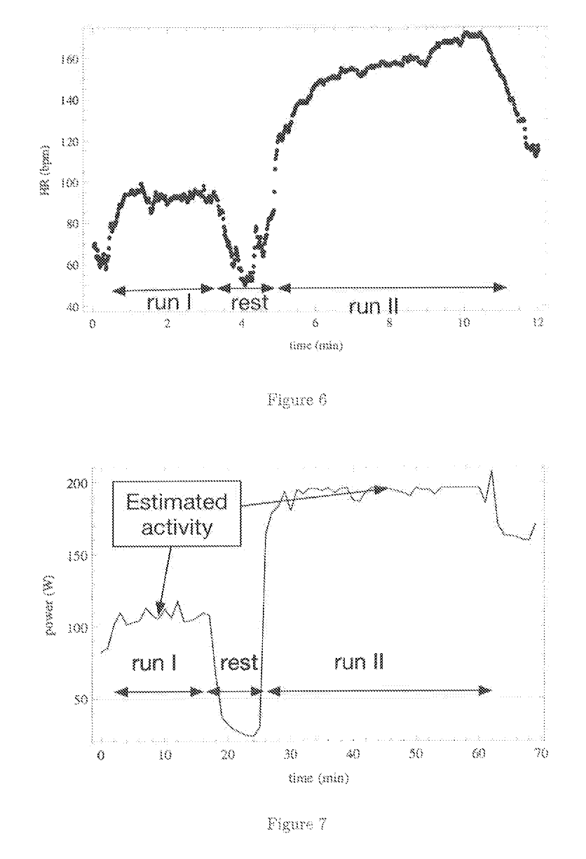

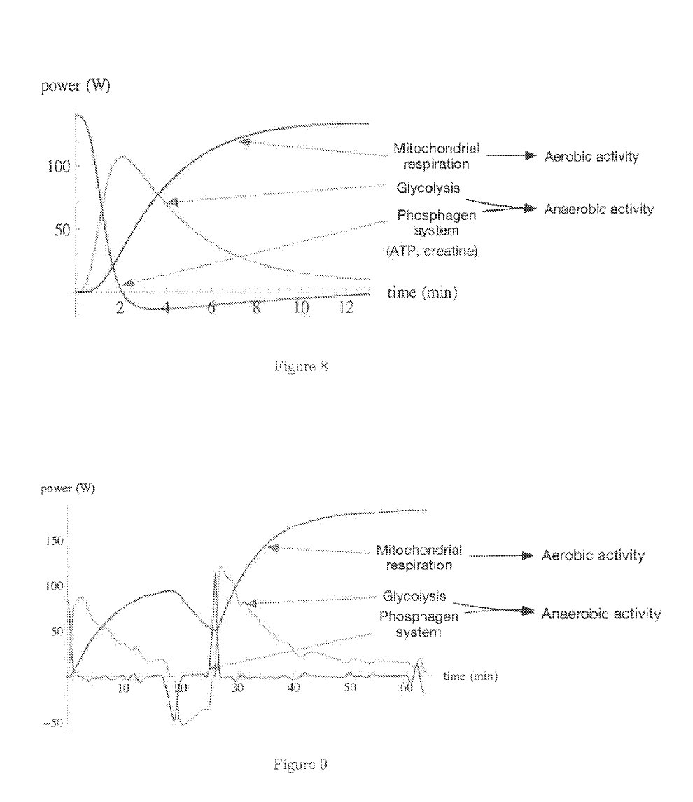

Up to this point, it has been demonstrated how a basic model that predicts dynamic changes in heart rate in response to different activity levels and thereby physiological loads can be used to either aid signal processing techniques in order to provide more accurate heart rate predictions or how it can be utilized to infer the physiological load for different exercise or rest states. A second use of this dynamic model includes using it in it's inverted form with HR predictions obtained from other algorithms. In FIG. 6 the HR obtained from an ECG-based device for two consecutive running sessions, the first being a shorter less intense run than the second, is depicted. Using the inverted dynamic heart rate model discussed earlier, it is possible to obtain an estimate for physiological load shown in FIG. 7, where two rectangular regions are shown for each running session, making clear the difference in time and intensity between the two exercises.

As outlined earlier, the current invention pertains to providing measurements of instantaneous activity levels as opposed to steady-state concepts such as the lactate threshold. It has already been demonstrated how estimates of instantaneous physiological load and thereby energy consumption can be obtained using measures of motion and heart rate activity. In this next section, the current invention further segments the estimated instantaneous activity level in terms of the different biochemical energy systems that contribute towards energy production in the body.

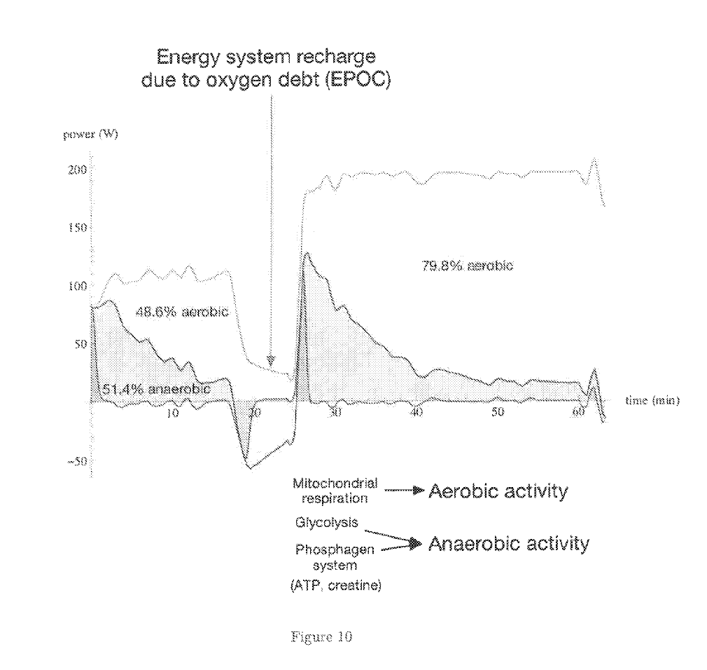

The energy system most directly linked to the muscle proteins that make movement possible is known as the phosphagen energy system. This group consists of molecules that can carry a high energy phosphate charge such as ATP and creatine-phosphate. Cells generally contain a tiny amount of these molecules, but can recharge them rapidly by breaking down glucose. The latter can be performed either in an oxygen dependent (aerobic respiration) or an oxygen independent manner (lactic fermentation). In the case of the latter, the glucose molecule is not broken down fully to CO₂, but is instead converted to lactic acid, for which the accumulation capacity is limited. It is possible to model these processes mathematically to produce estimates for the activity of each of these processes at different times and different physiological loads. In FIG. 8 the degree to which each system is engaged at different time points given a full physiological load (100%), using a simple ODE model of the system, is shown. Using the instant activity levels calculated for the two running sessions shown in FIG. 7 as the physiological load value in this model, it is possible to predict the contribution of each energy system as shown in FIG. 9. Note how the phosphagen system is quick to respond but is soon exhausted, while anaerobic glycolysis is second to be engaged with a larger capacity to sustain exercise. Finally the aerobic system is the slowest, but only sustainable energy source for extended exercise sessions. Note also how the slower aerobic energy system trajectory in FIG. 9 closely follows the trajectory of the HR shown in the HR data, FIG. 6, since HR is closely coupled to the rate at which the body can supply oxygen to the muscles.

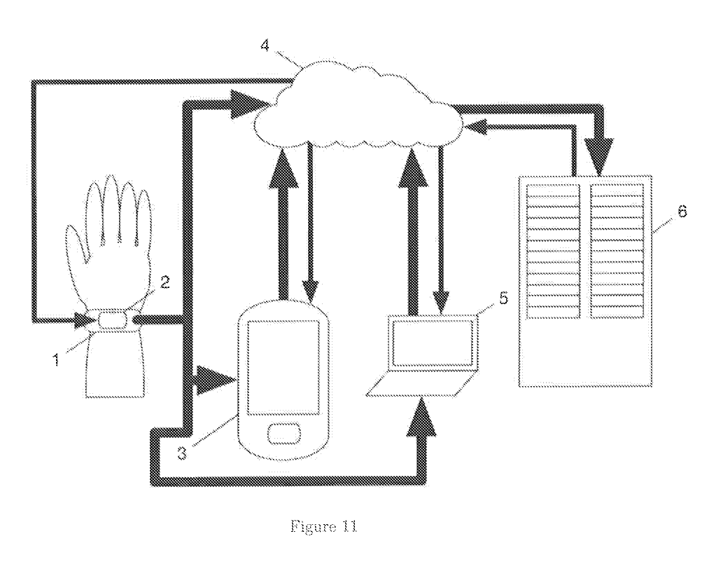

In FIG. 10 it is shown that the contribution of all three energy systems can be added together in such a way that the original physiological load estimated in FIG. 8 can be used to segment physiological load in terms of the contribution of each system. Note also how as expected the first brief run has a larger contribution from anaerobic energy systems than the longer sustained run and how between runs, the negative values for the phosphagen and anaerobic system fluxes indicate that the aerobic energy system is acting to recharge these reservoirs.

A basic embodiment of the inventions described above concerning motion compensated heart-rate calculation and instant physiological load estimation is demonstrated in FIG. 11, where 1 is a wearable electronic device containing the necessary sensor means to measure a pulse and motion signal. The wearable device optionally contains a display (2) and is capable of transmitting data to a mobile device (3) and or directly to an Internet based platform (4). The data can be stored and further processed on a server (6) for future retrieval and to be viewed on a computing platform exemplified by the personal computer (5), the mobile phone (3) and or wearable device (1).

What is claimed:

1. A method for augmenting heart rate predictions determined from a heart rate signal using a dynamic heart rate model, the method comprising:
(a) measurement of a motion signal from a motion capturing sensor;
(b) measurement of a heart rate signal from a heart rate sensor;
(c) application of a dynamic heart rate model which infers a heart rate from the motion signal and other parameters during periods when the heart rate signal is distorted;
(d) transmitting the heart rate.

2. The dynamic heart rate model of claim 1, which may comprise an ordinary differential equation (ODE) model.

3. The parameters of claim 1, which may be inferred in conjunction with a probabilistic framework, such as Hidden Markov Models.

4. A system for augmenting heart rate predictions determined from a heart rate signal using a dynamic heart rate model, the system comprising:
(a) a wearable device comprising a motion capturing sensor and a heart rate sensor;
(b) measurement of a motion signal from the motion capturing sensor which may comprise an accelerometer;
(c) measurement of a heart rate signal from the heart rate sensor which may comprise an electrocardiogram (ECG) or photoplethysmography (PPG) sensor;
(d) application of a dynamic heart rate model which infers a heart rate from the motion signal and other parameters during periods when the heart rate signal is distorted;
(e) transmitting the heart rate.

5. The dynamic heart rate model of claim 4, which may comprise an ordinary differential equation (ODE) model.

6. The parameters of claim 4, which may be inferred in conjunction with a probabilistic framework, such as Hidden Markov Models.

7. The system of claim 4 with the heart rate reported in its display

8. The system of claim 4, which can transmit the heart rate to a mobile electronic device, exemplified by a mobile phone.

9. The mobile electronic device of claim 8 configured to display the heart rate.

10. The system of claim 4 with the means to transmit the heart rate data wirelessly to a platform where said data can be stored, analyzed and viewed on client computing platforms, including but not limited to mobile computing devices, home computers or a wearable electronic device.

11. A method for inferring an instantaneous estimate of physiological load using a dynamic heart rate model, the method comprising:
(a) measurement of a motion signal from a motion capturing sensor;
(b) measurement of a heart rate signal from a heart rate sensor;
(c) the application of a dynamic heart rate model to estimate the instantaneous physiological load;
(e) transmitting the instantaneous physiological load estimate.

12. The dynamic heart rate model of claim 11, which may comprise an ordinary differential equation (ODE) model.

13. The parameters of claim 11, which may be inferred in conjunction with a probabilistic framework, such as Hidden Markov Models.

14. A system for inferring an instantaneous estimate of physiological load using a dynamic heart rate model, the system comprising:
(a) a wearable device comprising a motion capturing sensor and a heart rate sensor;
(b) measurement of a motion signal from the motion capturing sensor which may comprise an accelerometer;
(c) measurement of a heart rate signal from the heart rate sensor which may comprise an electrocardiogram (ECG) or photoplethysmography (PPG) sensor;
(d) the application of a dynamic heart rate model to estimate the instantaneous physiological load;
(e) transmitting the instantaneous physiological load estimate.

15. The dynamic heart rate model of claim 14, which may comprise an ordinary differential equation (ODE) model.

16. The parameters of claim 14, which may be inferred in conjunction with a probabilistic framework, such as Hidden Markov Models.

17. The system of claim 14 with the instantaneous estimate of physiological load reported on its display.

18. The system of claim 14, that transmits the instantaneous estimate of physiological load to a mobile electronic device, exemplified by a mobile phone or directly to a cloud platform.

19. The mobile electronic device of claim 18 configured to display the instantaneous estimate of physiological load.

20. The system of claim 14 with the means to transmit the physiological load estimate data wirelessly to a platform where said data can be stored, analyzed and viewed on client computing platforms, including but not limited to mobile computing devices, home computers or a wearable electronic device.

21. A method for calculating the relative contribution of different biochemical energy systems to the instantaneous physiological load, the method comprising:
(a) measurement of a motion signal from a motion capturing sensor;
(b) measurement of a heart rate signal from a heart rate sensor;
(c) the application of a dynamic heart rate model that infers heart rate from heart rate signals or motion signals and other parameters to estimate the instantaneous physiological load;
(d) calculation of the relative contribution of different biochemical energy systems to the instantaneous physiological load estimate;
(e) transmitting the relative biochemical energy system contribution to the instantaneous physiological load.

22. The dynamic heart rate model of claim 21, which may comprise an ordinary differential equation (ODE) model.

23. The parameters of claim 21, which may be inferred in conjunction with a probabilistic framework, such as Hidden Markov Models.

24. The energy systems of claim 23, which may be one or more of the following groups: phosphagen system, anaerobic glycolysis and aerobic respiration.

25. A system for calculating the relative contribution of different biochemical energy systems to the instantaneous physiological load estimate, the system comprising:
(a) a wearable device comprising a motion capturing sensor and a heart rate sensor;
(b) measurement of a motion signal from the motion capturing sensor which may comprise an accelerometer;
(c) measurement of a heart rate signal from the heart rate sensor which may comprise an electrocardiogram (ECG) or photoplethysmography (PPG) sensor;
(d) the application of a dynamic heart rate model to estimate the instantaneous physiological load;
(e) calculation of the relative contribution of different biochemical energy systems to the instantaneous physiological load estimate;
(f) transmission of the relative contribution of different biochemical energy systems to the instantaneous physiological load.

26. The dynamic heart rate model of claim 25, which may comprise an ordinary differential equation (ODE) model.

27. The parameters of claim 25, which may be inferred in conjunction with a probabilistic framework, such as Hidden Markov Models.

28. The energy systems of claim 25, which may be one or more of the following groups: phosphagen system, anaerobic glycolysis and aerobic respiration.

29. The system of claim 25 with the relative contribution of different biochemical energy systems to the instantaneous physiological load reported on its display.

30. The system of claim 25, that transmits the relative contribution of different biochemical energy systems to the instantaneous physiological load to a mobile electronic device, exemplified by a mobile phone or directly to a cloud platform.

31. The mobile electronic device of claim 25 configured to display the relative contribution of different biochemical energy systems to the instantaneous physiological load.

32. The system of claim 25 with the means to transmit the relative contribution of different biochemical energy systems to the instantaneous physiological load data wirelessly to a platform where said data can be stored, analyzed and viewed on client computing platforms, including but not limited to mobile computing devices, home computers or a wearable electronic device.
